(19) United States
(12) Patent Application Publication
(10) Pub. No.: US 2002/0004045 A1
(43) Pub. Date: Jan. 10, 2002

(54) Reduction of adipocyte number and adipose tissue depots in animals

(76) Inventor: Julio L. Pimentel, Buford, GA (US)

Correspondence Address: jULIO l. pIMENTEL, 3206 windgate dr, buford, GA, 30519, US

(21) Appl. No.: 09/819,099

(22) Filed: Apr. 4, 2001

Related U.S. Application Data
(63) Continuation of application No. 09/251,796, filed on Feb. 17, 1999, now abandoned.

(51) Int. Cl. A61K 39/395

(52) U.S. Cl. 424/141.1

(57) ABSTRACT

A method to decrease abdominal adipose and peripheral adipose depots in birds by in-ovo inoculation of avian antibodies against adipocyte or and specific adipocyte antigen determinants to 5-28 day-old embryos, or by orally feeding newborn mammals (within 24 hours of birth) with antibodies against adipocyte or and specific adipocyte antigen determinants.

### BACKGROUND OF THE INVENTION

1. Field of the Invention

A method to decrease the number of adipocyte and adipose tissue depots in animals specially animals for human consumption.

2. Discussion of the Background

The poultry and animal industry are concerned about the amount of abdominal and subcutaneous fat in the animals. This problem is not only a concern for the animal grower but also for the consumer. In the case of chickens, abdominal fat is trimmed which is either a cost for the processor or for the consumer. It is a cost for the poultry processor because they have paid for live weight and not for lean mass therefore that fat is a waste product; on the other hand, it is a cost for the consumer because fat is trimmed by the processor and the price increased or the fat is sold to the consumer and they have to trim and dispose of this undesirable waste product.

The poultry and animal industry are looking for ways to decreased abdominal and subcutaneous fat in animals. Several ways have been tried with variable results i.e. changing feeding programs, use of repartitioning agents, hormones and others.

Adipose tissue has an important function, which is to store energy, as triglycerides. Triglycerides are stored and then mobilized from adipocytes when the animal needs energy to respond to maintenance or other vital function (Cartwright et al 1991). As the bird grows, abdominal adipocyte cell number increases (hyperplasia) in function of an increase in body mass. As the percentage of abdominal fat increases, adipocyte cell volume increase (hypertrophy), or conversely, as adipocyte volume increase adiposity increases (Cartwright et al , 1988). Adipose tissue growth in chickens up to 16 weeks of age occurs by both hyperplasia and hypertrophy.

Modern commercial broiler chicks have been selected for increased body weight gain but this has brought about an increase in fat accumulation. When comparing commercial and non selected broilers of the same age or weight, the commercial broilers have more adipose tissue accumulation than the non-selected birds(Cartwright et al, 1988).

The efficacy of methods that reduce the number of adipocytes will depend on the ability of adipose tissue to replace reduced adipocytes or hypertrophy existing cells but in the case of chickens it has been observed that after partial lipectomy abdominal adipose tissue volume was not replaced (Cartwright et al , 1988).

Therefore destruction of existing adipocyte cells or manipulation of early proliferation of adipocyte can provide a method to effectively reduce carcass and abdominal fat and improve body composition.

### SUMMARY OF THE INVENTION

A method for the reduction of adipose tissue depots in animals.

### DESCRIPTION OF THE PREFERRED EMBODIMENTS

Newly hatched chicks have a fix number of pre-adipocyte cells (cells that will generate adipocyte and then form fat depots in the grown chick). These cells as the chick grows increase in number and size; by inhibiting or destroying these cells then the number of fat depots can decrease or disappear.

Preliminary work has been done using monoclonal antibodies to pig adipocytes. De Clerq, et al (1997) showed that monoclonal antibodies to preadipocytes destroyed pig adipocytes in vitro only when complement was added to the same culture. They also observed that injecting the same antibody (1 mg/kg body weight) to piglets resulted in a 20% reduction in subcutaneous and leaf fat lipids at 35 days of age. Wright et al (1990) developed a monoclonal antibody to adipocyte but they found cross-reactivity with other tissue cells. In a new study, Wright et al (1995) found that by injecting a pool of monoclonal antibodies against adipocytes to rats resulted in a 30% decrease in inguinal fat pad weight; this time the antibodies were specific to adipocytes.

Young growing rats treated with ip injection of sheep anti-adipocytes antibodies for four consecutive days had a reduction in adipose tissue depots and increased body weight. The reduction in adipose depots was due to a decrease in both cell number and size (Panton et al, 1990).

Passive immunization of lambs (three consecutive ip injection daily) with a horse anti-adipocyte antibody resulted in a decrease in perirenal adipose tissue weight and backfat thickness (Nassar and Hu, 1991).

Three U.S. patents (Flint 1992 a,b and Cryer et al, 1997) teach us that adipose tissue can be decreased by injecting an antigen specific to adipocytes (active immunity) or injecting the antibodies (passive immunity) in pigs, lambs and cows.

Butterwith et al (1989 and 1992) demonstrated that antibodies can be raised against chicken pre-adipocyte and adipocyte cells, but they failed to demonstrate true cytotoxic effect due to the antibodies. What they showed was the effect of the chicken immune system attacking their own adipose tissue because they had a foreign particle (mammalian antibody) attached to them. In other cases the need of complement was necessary to produce a cytotoxic effect.

Why do we want to in-ovo inoculate chicks with avian antibodies against adipocytes?

Newly hatched chicks are dependent on the maternal transfer of antibodies from the yolk. The IgG is transferred to the fetal serum of the embryo beginning on day 12 but is not until embryonic day 19-21 that larger amounts of IgG are transferred. For newly hatched chicks to produce IgM and IgG it take between 2 and 7 days post hatch and between 6 and 13 post hatch for IgA. (Kaspers et al, 1991).

In-ovo vaccination is currently a common practice in the poultry industry. More than 90% of hatcheries use in-ovo vaccination for diseases like Marek and New Castle. Several researchers have studied the effect of in-ovo inoculation of avian immunoglobulin for the protection against diseases, i.g. infectious bursal disease (Deshpande et al, 1996 and Eterradossi et al, 1997), rotavirus (Shaky et al, 1993 and 1994) and New Castle disease virus (Stone et al, 1992). It was observed that the inoculated antibodies could be detected up to 24 days after inoculation. The inoculated antibodies behave similarly to maternal IgG which have been detected in the circulatory system of turkey poults and chicks 3-4 weeks after hatching with a half-life of 3 days in chicks and 4.08 days in poults (Shawky et al, 1994).

Antibodies against New Castle disease virus that were in-ovo inoculated into the yolk sac of 18-day-chick embryos were found in serum 1 to 9 days post hatched. This suggests that no changes occurred in the transfer of the gammaglobulins from the yolk sac to serum. It was also reported that mammalian antibodies injected into the yolk sac of pigeons were not absorbed into the serum (Sander et al, 1998).

Reynolds (1998) teaches us an in-ovo method to produce passive immunity against diseases in birds but not to alter the body composition of the same.

Taking in consideration all the present literature and the lack of the same, a new and improved method has been developed, which is an avian antibody against a specific protein in the adipocyte to be in-ovo injected into 5-20-day chicken embryos. Since the antibody is of avian origin and administered to avians, their owns complement system will help in destroying adipocytes.

The same principle in this application can be used in mammals, with the difference that the antibody has to be of the same origin to which the decrease in fat depots wants to be performed; in other words cow to cow, or pig to pig. This has the objective to let the antibody call up the complement system to help destroy the adipocyte. By using their own complement fewer antibodies may be needed.

It will be apparent for those skilled in the art that the aforementioned objectives and other advantages may be further refined by the practice of the present invention.

The following steps will be required for the successful completion of the present application:

1.—Preparation of Adipocytes from Newly Hatched Chicks

Adipocyte will be prepared by collagenase digestion as described by Butterwith et al (1989). Adipose tissue will be collected from newly hatched chickens and minced with 10 ml Krebs Ringer phosphate buffer pH 7 containing 10 mg glucose, 3 gr. bovine serum albumin (BSA) and 10 mg collagenase and incubated for 70 min at 37° C. The digest will be filtered and washed with buffer and the floating adipocytes resuspended in Medium 199 containing 10 gr. BSA/1 before incubating for an additional 120 min at 37° C. The medium is then removed and 10 ml of extraction medium (50 mM Na₂HPO₃ pH 7, 10 mM ethyleneglycol-bis-(Beta-aminoethylether) N,N′-tetraacetic acid(EGTA), 1.25M sucrose) containing 0.2 mM phenyl-methylsulphonylfluoride is added and the suspension vortex for 2 min. The homogenate is centrifuge at 500×g for 5 minute to remove floating fat and the infranatant centrifuged at 100,000×g for 1 h. Membranes are resuspended in 50 mM Na₂HPO₄/10 mM EGTA pH 7.4 containing 400 g sucrose/l, and overlaid with 320 g sucrose/l followed by 50 mM Na₂HPO₄ pH 7.4/10 mM EGTA. Membranes are separated by spinning at 100,000×g for 1 h and resuspended in phosphate buffered saline pH 7.3 (PBS).

2.—Immunization of Rabbits

For immunizations, rabbits will receive 0.5 mg of membrane protein dissolved in 0.4 ml PBS and emulsified with 0.4 ml complete Freund's adjuvant. Subcutaneous injections will be given in the back of the rabbit in 8 sites and 100 ul per site. Secondary boosting will be 3 weeks after the first injection following the same protocol but this time the membrane suspension is emulsified with incomplete Freund's adjuvant. A third boost will be given if the antibody titer is determined to be low. Rabbits will be bled 1 week after the second injection.

3.—Testing for Antibody Production

An Elisa test will be developed for the determination of the antibody titer. Briefly, Elisa plates will be coated with 100 ul of a 10 ug/ml membrane protein and then blocked with 1.5% fish serum. Serum samples from a pre-immunization and post-immunization will be tested and compared to each other for the determination of the titer. Titer will be determined as the inverse dilution at with the absorbance of the immunized serum is double that of the control.

4.—Purification of Rabbit Anti-adipocyte Antibodies

Chicken adipocyte membrane protein will be coupled to an affinity chromatography gel and the hyperimmunized rabbit serum containing the antibody will be ran through the column in order to affinity purify the antibodies. This will have all the antibodies that bind to chicken adipocytes.

5.—Isolation of Chicken RBC, Liver, Muscle and Myelin Cells

Membrane protein from chicken red blood cells, liver, muscle and myelin will be isolated and bound to an affinity gel column. These membranes will be used for the binding of antibodies that can cross-react with chicken adipocytes.

6.—Specificity of Purified Antibody

The antibody purified from step 4 will be sequentially passed through the four different columns containing the other membranes (step 5). The antibody not bound to those columns will be the specific antibody to adipocytes that will not crossreact with other cell in the chicken. The purpose of this step is to bind the antibodies that crossreact with the adipocyte. In other words, it will be the specificity step.

7.—Binding Specific Antibody and Purification of Specific Chicken Adipocyte Protein(s)

The antibody not bound (resulting from step 6) which is specific to chicken adipocytes will be bound to an affinity column. The adipocyte membrane extract (step 1) will be passed through this column to purify the specific protein(s). This purified protein(s) specific to adipocytes will be used for the production of antibodies in the hen.

8.—Antibody Production in Hens

Antibody production in hens is a common practice and several patents teach the effect of those antibodies in animals. Sharp (1997) and Crisostomo et al (1997) show that auto-immunization is a workable idea since immunization against vasoactive intestinal polypeptide (VIP) suppressed prolactin secretion and inhibited broodiness in turkeys. The protein(s) obtained in step 7 will be used for antibody production in the hen. The molecular weight of this protein will be determined first to see if binding to a carrier protein is needed. Briefly, adult hens will be im injected with 100 ug of the protein(s) or the coupled protein(s) dissolved in 0.2 ml PBS and emulsified with 0.2 ml Freund's complete adjuvant. Booster injections emulsified in incomplete adjuvant will be 2 and 4 weeks after the first injection. The need for booster injections will depend in the titer of the antibodies present in the egg yolk of the hyperimmunized hen. Egg collection will start 1 week after the second injection.

9.—Titer Determination

The antibody titration will be determined by an Elisa test. Briefly, 100 ul of a 10 ug protein/ml will be added to each well in the plate and after 18 hours incubation, 1.5% fish serum in PBS will be used to block the Elisa plate. Egg samples from preimmunization and post-immunization will be tested and compare to each other for titer determination.

10.—Antibody Purification

There are several procedures for the purification of antibodies from egg yolk, using organic solvents, polyethylene glycol, water and others. All of them result in good antibody yield.

11.—In-ovo Dose Response Study

Once the antibodies have been purified and the titer determined, the antibody will be diluted into three concentrations, high, medium and low. Two hundred fifty 18-day old embryos divided into five treatments of fifty each will be in-ovo injected with 100 ul of the antibody suspension (high, medium and low), phosphate buffered saline (control) and non-injected control. Chicks will be hatched and raised as commercially done.

12.—Grow-out Study

Newly hatched chicks from each of the high, medium, low, PBS injected and non-injected control treatments will be separated into four repetitions of 10 chicks each and fed a commercial diet. At 3 weeks of age half and at 7 weeks of age the other half will be killed for the determination of adipose tissue. Weekly body weight and fed intake will be monitored in order to determine feed conversion (feed intake/body weight gain).

13.—Determination of Optimum Antibody Level

From the grow-out study we can determine the optimum dose required for the reduction of adipose tissue and the other parameters measured. Since there is going to be a destruction of adipocytes, an increase of plasma triglycerides is expected. This could affect intake and the need for excess energy in the diet, which will require new feed formulation for the maximum economical advantage.

14.—Other Methods to Purify Specific Adipocyte Protein(s) and Antibodies

The antibody can be purified by absorbing the crude antibody produced in rabbits against cells from liver, muscle, myelin, and red blood cells. Absorption is carried by incubating the rabbit antibody and the respective cells for a set period of time (30-120 min.). Antibodies that are not specific to those cells will not bind and will remain in suspension after centrifugation. Then that purified antibody is bound to the column to isolate the protein specific to chicken adipocytes.

15.—Other Animal Uses

This proposed method could also be used in mammals. The antibody production, protein purification and specific antibody will be the same as described above. It has to be taken into consideration that the antibody to be inoculated into the animal has to be produced in the same animal specie; this is to allow the complement to help destroying the adipocytes. The antibodies are not injected but orally fed. Newborn mammals have the capability to absorb intact protein through the intestine within the first 24 hour from birth. This allows the immunoglobulins to be absorbed and go into circulation. The antibodies to be inoculated will behave as antibodies present in colostrum.

It will be apparent for those skilled in the art that a number of modifications and variations may be made without departing from the scope of the present invention as set forth in the appending claims.

### REFERENCES

1) Butterwith S C, S Kestin, H D Griffin, J Beattie and D J Flint, 1992. Identification of chicken (gallus domesticus) adipocyte plasma membrane and differentiation specific proteins using SDS-PAGE and western blotting. Comp. Biochem. Physiol. 101B(# 1/2): 147-151.

2) Butterwith S C, S Kestin, H D Griffin and D J Flint, 1989. Cytotoxic antibodies to chicken adipocytes and their precursors: lack of tissue specificity. British Poultry Science 30: 371-378.

3) Cartwright A L, H L Harms and D R Campion, 1988. Adipose cellularity in non-selected and selected broiler stocks: measurements at equal weights and age. Poultry Science 67: 1338-1344.

4) Cartwright, A L, 1991. Adipose cellularity in Gallus domesticus: investigations to control body composition in growing chickens. J. Nutr. 121: 1486-1497.

5) Crisostomo S, D Guemene, M Garreau and D Zadworny, 1997.

Prevention of the expression of incubation behavior using passive immunization against prolactin in turkey hens (Melleagris gallopavo). Reprod. Nutr. Dev. 37: 253-266.

6) Cryer, A, D Flint and S Kestin, 1997. U.S. Pat. No. 5631009. Porcine adipocyte antigens and their use in the immunological control of fat.

7) De Clerq, L et. al. (1997). An anti-adipocyte monoclonal antibody is cytotoxic to porcine pre-adipocytes in vitro and depresses the development of pig adipose tissue. J. Anim. Sci. 75: 1791-1797

8) Deshpande M S and L Muniyappa, 1996. Evaluation of maternally derived antibody activity against infectious bursal disease virus in chickens. Indian Veterinary J. 73: 249-252.

9) Eterradossi N, D Toquin, H Abbassi, G Rivallan, J P Cotte and M Guillet, (1997). Passive protection of specific pathogen free chicks against infectious bursal disease by in ovo injection of semi-purified egg yolk antiviral immunoglobulin. J. Vet Medicine-Series B 44: 371-383.

10) Flint, D, 1992a. U.S. Pat. No. 5096706. Antigen based treatment for adiposity.

11) Flint, D, 1992b. U.S. Pat. No. 5102658. Antibody treatment for adiposity.

12) Flint, D , 1992. Immunological manipulation of adiposity. Proc. Nutr. Soc. 51: 433-439.

13) Kaspers B, I Schranner and U Losch, 1991. Distribution of immunoglobulins during embryogenesis in the chicken. J. Vet. Med. A. 38: 73-79.

14) Nassar, A H and C Y Hu, 1991. Growth and carcass characteristics of lambs passively immunized with antibodies developed against ovine adipocyte plasma membranes. J. Anim. Sci. 69: 578-586.

15) Panton, D, C Futter, S Kestin and D Flint, 1990. Increased growth and protein deposition in rats treated with antibodies to adipocytes. Am. J. Physiol. 258: E985-E989.

16) Reynolds, D, 1998. U.S. Pat. No. 5807551. Method to provide artificial passive immunity in birds.

17) Sander, J E, E M Willinghan, J L Wilson and S G Thayer, 1998. The effect of inoculating enterococcus faecalis into the yolk sac on chick quality and maternal antibody absorption. Avian Diseases 42: 359-363.

18) Sharp, P, 1997. Immunological control of broodiness. World's Poultry Sci. 53: 23-31.

19) Shawky, S, Y M Saif and D E Swayne, 1993. Role of circulating maternal anti-rotavirus IgG in protection of intestinal mucosal surface in the turkey poults. Avian Dis. 37: 1041-1050.

20) Shawky, S A, Y M Saif and J McCormick, 1994. Transfer of maternal anti0-rotavirus IgG to the mucosal surfaces and bile of turkey poults. Avian Diseases 38: 409-417.

21) Stone, H D, M Brugh and Z Xie, 1992. Simulation of maternal immunity by inoculation of immune yolk preparations into the yolk sac of 1 day old embryo. Avian Diseases 36: 1048-1051.

22) Wright J T and G J Hausman, 1990. Adipose tissue development in the fetal pig examined using monoclonal antibodies. J Anim Sci 68: 1170-1175.

23) Wright J T and G J Hausman, 1995. Monoclonal antibody-mediated cytotoxicity of adipocyte. Obesity Research 3: 265-272.

What is claimed is:

1. A method to reduce adipose tissue depots in birds by in-ovo inoculation of avian antibodies against adipocyte antigen determinants.

2. A method of claim 1 wherein adipose tissues are inside and/or around the abdominal cavity or underneath the skin covering the body of the bird.

3. A method of claim 1 wherein birds are for human consumption, which includes chickens, hens, roasters, capons, turkeys, quails, ducks, pheasants and pigeons.

4. A method of claim 1 wherein in-ovo inoculation is done between 5 and 21 days of embryonic development in the chicken, from 5-28 days in turkeys, ducks, pheasants and from 5-18 for quails and pigeons.

5. A method of claim 1 wherein in-ovo inoculation is by puncturing the egg shell and shell membrane with a needle and depositing the antibody in the interior either in the white or in the yolk, preferably into the yolk.

6. A method of claim 1 wherein avian antibodies, extracted from the serum or egg, is produced in the same specie of avian to which the antibody will be administered.

7. A method in claim 1 wherein the antibody can be engineered or synthesized; monoclonal, bacterial, plant or yeast; in such a form that can activate the animal's own complement system.

8. A method of claim 1 wherein adipocyte antigen determinants are structural components and are purified from the adipocyte plasma membrane.

9. A method of claim 1 wherein adipocyte antigen determinants are chemically synthesized.

10. A method of claim 1 wherein reduction of adipocyte cell mass is due to the killing mediated by the antibody administered and the complement from the animal to which the antibody is administered.

11. A method to reduce adipose tissue depots in mammals by orally fed antibodies specific to adipocyte antigen determinants.

12. A method in claim 11 wherein adipose tissue are inside and/or around the abdominal cavity or underneath the skin covering the body of a mammal i.e. backfat.

13. A method in claim 11 wherein mammals comprise animals for human consumption that includes: dairy cattle, beef cattle, pigs, lambs and rabbits.

14. A method in claim 11 wherein the antibody, from serum or colostrum, is produced in the same animal specie to which the antibody will be administered.

15. A method of claim 11 wherein the antibody can be engineered or synthesized; monoclonal, bacterial, plant or yeast; in such a form that can activate the animal's own complement system.

16. A method in claim 11 wherein the antibody is orally administered within 24 hour of birth.

17. A method of claim 11 wherein adipocyte antigen determinants are structural components and are purified from the adipocyte plasma membrane.

18. A method of claim 11 wherein adipocyte antigen determinants are chemically synthesized.

19. A method of claim 11 wherein reduction of adipocyte cell mass is due to the killing mediated by the antibody administered and the complement from the animal to which the antibody is administered.
